(19) United States
(12) Patent Application Publication
(10) Pub. No.: US 2024/0336962 A1
(43) Pub. Date: Oct. 10, 2024

(54) A Method of Amplification of a Nucleic Acid

(71) Applicant: Agency for Science, Technology and Research, Singapore (SG)

(72) Inventors: Lian Chye Winston Koh, Singapore (SG); Kiat Whye Kong, Singapore (SG)

(21) Appl. No.: 18/294,286

(22) PCT Filed: Aug. 5, 2022
(86) PCT No.: PCT/SG2022/050560

§ 371 Date: Feb. 1, 2024

(30) Foreign Application Priority Data
Aug. 5, 2021 (SG) 10202108592Y

(51) Int. Cl. C12Q 1/6851; C12Q 1/6806; C12Q 1/6876

(52) U.S. Cl. CPC C12Q 1/6851; C12Q 1/6876; C12Q 1/6806

(57) ABSTRACT

This disclosure relates to a method of amplification of a target nucleic acid, the method comprising subjecting the target nucleic acid to one or more amplification step in the presence of a mixture comprising a control nuclei acid, a surfactant and an oligonucleotide primer and/or probe capable of hybridizing with the target nucleic acid, wherein the oligonucleotide primer and/or probe comprises a cleavage site and a cleavable 3′ end. In one embodiment, the target nucleic acid is cell-free RNA and the method comprises annealing the target nucleic acid and performing a reverse transcription prior to the one or more amplification step. Also disclosed are nucleic acid amplification mixtures, kits for amplifications, and methods of detecting and/or determining the presence and/or the amount of a target nucleic acid.

### TECHNICAL FIELD

The present disclosure relates broadly to a method of amplification of a nucleic acid. In particular, the present disclosure relates to the amplification and quantification of a cell free nucleic acid.

### BACKGROUND

Due to their ability to detect small amounts of target molecules, nucleic acid amplification technologies have become fundamental tools in molecular diagnostics. One such tool is the Polymerase Chain Reaction (PCR), which is used for applications such as identification of pathogens, characterization of genetic disorders, identification of disease biomarkers, gene expression studies, and many others.

Variations of PCR have been developed to attempt to overcome the disadvantages of PCR which include for example poor precision, low sensitivity, and post-PCR analysis. Variations of PCR include for example, quantitative or real time PCR, isothermal PCR, immuno-PCR, and many others.

Real-time PCR detects a single copy of a transcript and requires much less RNA template than other methods. Real-time PCR measures the amount of PCR products during the exponential phase and do not require post-PCR analysis, as compared to traditional PCR which measures amplified fragments at the plateau phase post-PCR. However, real-time PCR requires separate priming reactions for each target and therefore it is not possible to return to the same preparation and amplify other targets at a later stage. Other disadvantages of real-time PCR include expensive equipment, reagents, and the requirement of sound experimental design and normalization techniques for accurate conclusions.

Other methods of amplification that are known in the art include methods such as the loop mediated isothermal amplification (LAMP) and strand-displacement amplification (SDA). These techniques can detect targets of interest such as pathogens or analytes at low concentrations (i.e. femto and picomolar concentrations). However, isothermal nucleic acid amplification lacks technical maturity and widespread application or commercialization under kit formats. Furthermore, most of the methods in isothermal nucleic acid amplification generates false positives and false negatives with high background noise, due to the low amplification temperature. Isothermal nucleic acid amplification also faces challenges such as complex primer design and requirement for various enzymes and denaturing agents.

Another known amplification technique is the immuno-PCR, which uses minimum amount of sample and allows detection of rare biomarkers in complex biological samples. However, immuno-PCR produces high background signals that prohibit meaningful results. The mechanism also involves complex conjugation chemistries to link the antibody and DNA-markers.

In summary, methods currently available in the art are known to have challenges in generating amplicons with high specificity and low background noise. In addition, some of the methods require complex primer design or multiple enzymes or reagents, requiring separate priming reactions for each target. Therefore, there is a need to provide an alternative method of amplifying a nucleic acid.

### SUMMARY

In one aspect, the present invention provides a method of amplification of a target nucleic acid, the method comprising annealing the target nucleic acid in the presence of a control nucleic acid, and subjecting the target nucleic acid to one or more amplification step in the presence of a mixture comprising a surfactant and an oligonucleotide primer and/or probe capable of hybridizing with the target nucleic acid, wherein the oligonucleotide primer and/or probe comprises a cleavage site and a cleavable 3′ end.

In some examples, wherein the amplification step of the nucleic acid is performed in the presence of three parts surfactant to one part amplification mixture.

In some examples, wherein the method comprises two amplification steps.

In some examples, wherein the method further comprises a step of freeze and thawing the amplified mixture.

In some examples, wherein the method further comprises a step of freeze and thawing the amplified mixture between the one or more amplification steps.

In some examples, wherein the amplification step includes interposing an annealing step between denaturation and priming.

In some examples, wherein the oligonucleotide primer and/or probe capable of hybridizing with the target nucleic acid comprises a 5′ end of a functional primer, a cleavage site, one or more matching DNA bases, and one or more mismatch DNA base with one or more blocking group at the 3′ end.

In some examples, wherein the cleavage site is one or more RNA residues.

In some examples, wherein the oligonucleotide primer and/or probe capable of hybridizing with the target nucleic acid comprises a 5′ end of a functional primer, a cleavage site consisting of one or more RNA residue, one or more matching DNA bases, and one or more mismatch DNA base with one or more blocking group at the 3′ end.

In some examples, wherein the method comprises the step of cleaving the oligonucleotide primer and/or probe with an RNase enzyme.

In some examples, wherein the control nucleic acid is added to the sample at a constant amount to thereby normalizes of the amplification efficiency across a plurality of samples, optionally the control nucleic acid is added to the sample at about 102 to 1010 copies.

In some examples, wherein the method further comprises a reverse transcription of the target nucleic acid after annealing step.

In some examples, wherein the method further comprises a step of quantifying the amount of target nucleic acid present in the sample and/or sequencing the target nucleic acid in the sample.

In some examples, wherein the target nucleic acid is a cell free nucleic acid, optionally a circulating cell free nucleic acid.

In some examples, wherein the target nucleic acid is a cell free RNA, optionally a circulating cell free RNA.

In some examples, wherein the target nucleic acid is obtained from a biological sample.

In some examples, wherein when the target nucleic acid is an RNA, the method comprises annealing the target nucleic acid is in the presence of a reverse primer of the target nucleic acid and the control nucleic acid, subjecting the annealed sample to reverse transcription, and subjecting the target nucleic acid to one or more amplification step in the presence of a mixture comprising a surfactant and an oligonucleotide primer and/or probe capable of hybridizing with the target nucleic acid, wherein the oligonucleotide primer and/or probe comprises a cleavage site and a cleavable 3′ end.

In some examples, wherein the method is a real time amplification method.

In another aspect, the present invention provides a nucleic acid amplification mixture comprising a first mixture comprising: a control nucleic acid, and a second mixture comprising: a surfactant, and an oligonucleotide primer and/or probe capable of hybridizing with a target nucleic acid, wherein the oligonucleotide primer and/or probe comprises a cleavage site and a cleavable 3′ end.

In yet another aspect, the present invention provides a method of detecting and/or determining the presence and/or the amount of a target nucleic acid comprising annealing the target nucleic acid in the presence of a control nucleic acid, and subjecting the target nucleic acid to one or more amplification step in the presence of a mixture comprising a surfactant and an oligonucleotide capable of hybridizing to the target nucleic acid comprising one or more RNA base and a cleavable 3′ end.

### Definitions

As used herein, the term “biological sample” refers to a sample obtained from a biological subject, including a sample of biological tissue or fluid origin obtained in vivo or in vitro. Hence, a “biological sample” may be a solid biological sample or a liquid biological sample. Examples of a “solid biological sample” may include biopsies, such as an organ biopsy, a tumor biopsy, stools, cell culture, food, plant extracts, and the like. Examples of a “fluid biological sample” or “liquid biological sample” include blood, serum, plasma, sputum, lavage fluid (for example peritoneal lavage), cerebrospinal fluid, urine, vaginal discharge, semen, sweat, tears, saliva, and the like. As used herein, the terms “blood”, “plasma”, and “serum” encompass fractions or processed portions thereof. Similarly, where a sample is taken from a biopsy, swab, smear, etc., the “sample encompass a processed fraction or portion derived from the biopsy, swab, smear, etc.

As used herein, the term “detecting” includes the step of determining the presence and/or absence of cfRNA. In some examples, the term “detecting” may further include the step of quantification of the cfRNA detected in the sample.

As used herein, the term “isolated” refers to a nucleic acid that is removed from its natural environment. An “isolated” nucleic acid is typically partially purified. As used herein, the term “nucleic acid” refers to a nucleotide sequence that typically includes nucleotides comprising an A, G, C, T or U base. In some examples, nucleotide sequences may include other bases such as inosine, methylcytosine, hydroxymethylcytosine, methylinosine, methyladenosie and/or thiouridine, and the like. The term “nucleic acid” may include both single and/or double stranded deoxyribonucleic acid (DNA) and ribonucleic acid (RNA), including environmental DNA (eDNA), genomic DNA, bacterial DNA, viral DNA, cell-free DNA (cfDNA), complementary RNA (cRNA), messenger RNA (mRNA), transfer RNA (tRNA), microRNA (miRNA), cell free RNA (cfRNA), circulating tumour RNA (ctRNA), bacterial RNA, viral RNA, ribosomal RNA (rRNA) and the like.

As used in herein, the term “target nucleic acid” refers to nucleic acid whose presence is to be detected or measured or whose function, interactions or properties are to be studied. Therefore, a target nucleic acid includes essentially any nucleic acid for which a detectable probe (e.g., oligonucleotide probe) or assay exists, or can be produced or isolated by one skilled in the art. Target nucleic acid may include disease markers, viral DNA and/or RNA, bacterial DNA and/or RNA, tumor markers, and the like.

As used herein, the term “real time” refers to the actual time during which a process or event occurs and/or tracking of temporal changes and/or trajectories of cellular changes in samples drawn from different time points.

As used herein, the term “surfactant” refers to a composition that stabilizes water-in-oil droplets that is capable of or that can encapsulate nucleic acids (such as DNA, cDNA, cfDNA, RNA, cfRNA, and the like). In some examples, the surfactant may comprise a particular repeat unit comprising a perfluoropolyether and a polyalkylene oxide unit. In some examples, the surfactant may be one or more of fluorosurfactant, non-ionic surfactant, anionic surfactant, cationic surfactant, amphoteric surfactant, and the like. In some examples, the fluorosurfactant may be synthesized by coupling oligomeric perfluorinated polyethers (PFPE) with polyethyleneglycol (PEG).

The term “micro” as used herein is to be interpreted broadly to include dimensions from about 1 micron to about 1000 microns.

The term “nano” as used herein is to be interpreted broadly to include dimensions less than about 1000 nm.

The terms “coupled” or “connected” or “attached” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween. For example, the cleavage compound as described herein cleaves the oligonucleotide (e.g. primer, probe, and the like) within or adjacent to the cleavage domain. Thus, the term “adjacent” means that the cleavage compound cleaves the oligonucleotide at either the 5′-end or the 3′ end of the cleavage domain. In some examples of the present disclosure, the cleavage reactions yield a 5′-phosphate group and a 3′—OH group.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. As used herein, the term “substantially no” or “very low” refers to a sequence homology of less than at least 20%, or 19%, or 18%, or 17%, or 16%, or 15%, or 14%, or 13%, or 12%, or 11%, or 10%, or 9%, or 8%, or 7%, or 6%, or 5%, or 4%, or 3%, or 2%, or 1%, or 0.9%, or 0.8%, or 0.7%, or 0.6%, or 0.5%, or 0.4%, or 0.3%, or 0.2%, or 0.1%, or 0.01% sequence homology to the target nucleic acid (for example any human gene). In some examples, the term “substantially no” or “very low” sequence homology refers to the control gene having substantially different sequence to the target nucleic acid (for example any human gene). In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of methods of amplifying and/or quantifying a nucleic acid are disclosed hereinafter. Also disclosed are methods of detecting and/or determining the presence and/or the amount of a target nucleic acid.

In one aspect, there is provided a method of amplification of a target nucleic acid, the method comprising annealing the target nucleic acid in the presence of a control nucleic acid, and subjecting the target nucleic acid to one or more amplification step in the presence of a mixture comprising a surfactant and an oligonucleotide primer and/or probe capable of hybridizing with the target nucleic acid, wherein the oligonucleotide primer and/or probe comprises a cleavage site and a cleavable 3′ end.

In some examples, the amplification step of the nucleic acid is performed in the presence of three parts surfactant to one part amplification mixture. In some examples, the amplification step is performed in an emulsion mixture. The emulsion mixture is made up of 1 to 10 parts of surfactant with 1 to 5 parts of amplification mixture, or 1 part of surfactant with 1 part of amplification mixture, or 2 parts of surfactant with 1 part of amplification mixture, or 3 parts of surfactant with 1 part of amplification mixture, or 4 parts of surfactant with 1 part of amplification mixture, or 5 parts of surfactant with 1 part of amplification mixture, or 6 parts of surfactant with 1 part of amplification mixture, or 7 parts of surfactant with 1 part of amplification mixture, or 8 parts of surfactant with 1 part of amplification mixture, or 9 parts of surfactant with 1 part of amplification mixture, or 10 parts of surfactant with 1 part of amplification mixture, or 3 parts of surfactant with 2 parts of amplification mixture, or 4 parts of surfactant with 2 parts of amplification mixture, or 5 parts of surfactant with 2 parts of amplification mixture, or 6 parts of surfactant with 2 parts of amplification mixture, or 7 parts of surfactant with 2 parts of amplification mixture, or 8 parts of surfactant with 2 parts of amplification mixture, or 9 parts of surfactant with 2 parts of amplification mixture, or 10 parts of surfactant with 2 parts of amplification mixture. In some examples, the emulsion is made up of 3 parts of surfactant with 1 part of amplification mixture. That is, the emulsion is made up of 3 parts of surfactant with 1 part of amplification mixture, when the amplification mixture is 10 μL, the surfactant is 30 μL, to thereby provide a total of 40 μL of emulsion mixture.

In some examples, the surfactant may be used at about 1% (w/w) to about 15% (w/w), or about 2, 3, 4, 5, 6, 7, 8, 9, 10, 11, 12, 13, 14, or 15% (w/w). In some examples, the surfactant may be 10% (w/w) of fluorosurfactant.

In some examples, method comprises two amplification steps.

In some examples, the method comprises 2, or 3, or 4, or 5 amplification steps. In some examples, the amplification cycle or step is repeated two times to five times. In some examples, the amplification step is repeated two times, or three times, or four times, or five times, or more. In some examples, the amplification step is repeated two times (i.e. two amplification cycles).

In some examples, the method further comprises a step of freeze and thawing the amplified mixture.

In some examples, wherein the method further comprises a step of freeze and thawing the amplified mixture between the one or more amplification steps.

In some examples, the freeze and thawing step may be referred to as the emulsion breaking step. The inventors of the present disclosure found freeze thawing the emulsion PCR product advantageously provides for a robust, non-chemical based method of recovering the emulsion PCR product.

In some examples, the step of freezing comprises subjecting the mixture to a condition that freezes the mixture to a solid state. For example, the step of freezing subjects the mixture to a below freezing conditions. In some examples, the method comprises the step of freezing the mixture to 0° C. to −100° C., or to −50° C., or to −60° C., or to −70° C., or to −80° C. or to −90° C., or to −100° C. In some examples, the method comprises the step of freezing the mixture to −80° C.

In some examples, the method comprises freezing the reaction mixture for 0.5 hour to overnight. In some examples, the method comprises freezing the reaction mixture for 0.5 hour, or 1 hour, or 1.5 hour, or 2 hours, or 2.5 hours, or 3 hours, or 3.5 hours, o 4 hours, or 4.5 hours, or 5 hours, or 6 hours, or 7 hours, or 8 hours, or 9 hours, or overnight.

In some examples, the step of thawing comprises subjecting the mixture to a condition that allows the mixture to gain heat and change to a liquid state. For example, the step of thawing subjects the mixture to an above 0° C. conditions. In some examples, the step of thawing subjects the mixture to room temperature condition, for example from about 0° C. to about 40° C.

In some examples, the method may comprise adding an amplification (e.g. PCR) mixture to the target nucleic acid. In some examples, the mixture may comprise a DNA polymerase, a dNTP mixture, a cofactor (such as Magnesium Chloride), an rhPCR mixture of the target nucleic acid, and an RNase (such as an RNase H2 enzyme).

In some examples, the method comprises generating an emulsion by adding 3 parts of surfactant to 1 part of PCR reaction mixture. In some examples, the method comprises mixing (such as vortexing) the emulsion generated until uniform turbidity. In some examples, the amplification step may be a thermocycling reaction with enzyme activation, denaturation, annealing, and extension.

In some examples, the method comprises freezing the reaction mixture for 1 hour before thawing at room temperature. In some examples, the freeze thaw is performed between each amplification (thermocycling) step. In some examples, the method comprises transferring the top fraction of the reaction mixture to a fresh tube. In some examples, the method further comprises topping up the fraction recovered with the same amount of polymerase (such as Taq polymerase) and RNase enzyme (such as RNase H2 enzyme) as used in the preceding PCR reaction.

In some examples, the method comprises a subsequent amplification step (e.g. a second or third or more thermocycling reactions) with enzyme activation, denaturation, hybridization, annealing and extension. In some examples, the method further comprises removing residual primers with an enzyme, followed by enzyme inactivation.

In some examples, the oligonucleotide primer and/or probe capable of hybridizing with the target nucleic acid comprises a 5′ end of a functional primer, a cleavage site, one or more matching DNA bases, and one or more mismatch DNA base with one or more blocking group at the 3′ end.

In some examples, the primer may comprise about 10 to 40 bases, or 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, 30 bases, 35 bases, or 40 bases.

In some examples, the functional primer may be about 16 to 24 bases, or about 16, 17, 18, 19, 20, 21, 22, 23, or 24 bases. In some examples, the reverse primers may comprise about 15 to 18 bases, or about 15, 16, 17, 18, 19, 20, 21, 22, 23, or 24 bases. In some examples, the qPCR primers may comprise about 15 to 20 bases, or 15, 17, 18, 19, 20, 21, 22, 23, or 24 bases

In some examples, the cleavage site is one or more RNA residue. In some examples, the cleavage site may comprise 2, 3, or 4 RNA residue. In some examples, the cleavage site is a single RNA residue or one RNA residue. In some examples, the cleavage site may be one or more of rU, rC, rG, or rA. In some examples, the cleavage site may be one of rC, rG, or rA.

In some examples, the cleavage site is one or more RNA residues.

In some examples, oligonucleotide primer and/or probe capable of hybridizing with the target nucleic acid comprises a 5′ end of a functional primer, a cleavage site consisting of one or more RNA residue, one or more matching DNA bases, and one or more mismatch DNA base with one or more blocking group at the 3′ end.

In some examples, the method comprises the step of cleaving the oligonucleotide primer and/or probe with an RNase enzyme.

In some examples, the cleavage site is cleaved by RNase H2 enzyme.

In some examples, the cleavage of the RNA residue releases the blocking group.

In some examples, the one or more matching DNA bases may comprise 1 DNA base, 2 DNA bases, 3 DNA bases, 4 DNA bases, 5 DNA bases, 6 DNA bases, 7 DNA bases, 8 DNA bases, 9 DNA bases, or 10 DNA bases. In some examples, the one or more matching DNA bases may be at the 3′ end of the cleavage site.

In some examples, the one or more mismatching DNA base may comprise 1 DNA base, 2 DNA bases, 3 DNA bases, 4 DNA bases, 5 DNA bases, 6 DNA bases, 7 DNA bases, 8 DNA bases, 9 DNA bases, or 10 DNA bases. In some examples, the one or more mismatching DNA base may be at the 3′ end of the matching DNA bases. In some examples, the primer may comprise one mismatching DNA base at the 3′ end of the primer.

In some examples, the primer and/or probe may comprise one or more blocking group. In some examples, the primer and/or probe may comprise 1, 2, 3, 4, 5, or more blocking groups. In some examples, the primer and/or probe may comprise 1 blocking group. In some examples, the primer and/or probe may comprise 2 blocking groups.

As disclosed herein, the blocking group may be a chemical moiety that is bound to the primer or other oligonucleotide such that an amplification reaction does not occur. For example, primer extension and/or DNA ligation does not occur. Once the blocking group is removed from the primer or other oligonucleotide, the oligonucleotide is capable of participating in the assay for which it was designed (e.g. PCR, ligation, sequencing, etc). Thus, the blocking group can be any chemical moiety that inhibits recognition by a polymerase or DNA ligase. The blocking group may be incorporated into the cleavage domain but is generally located on either the 5′- or 3′-side of the cleavage domain. In some examples, the blocking group is on the 3′ end of the oligonucleotide. The blocking group can be comprised of more than one chemical moiety. In the present invention the “blocking group” is typically removed after hybridization of the oligonucleotide to its target sequence. In some examples, the blocking group may be a C3 spacer (a phosphoramidite,

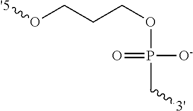

(for incorporation at 5′ end or internally), oror

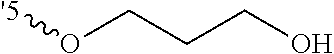

(for incorporation at 3′ end)), a hexanediol (a six carbon glycol spacer or

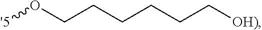

a 1′2′-dideoxyribose (dSpacer or

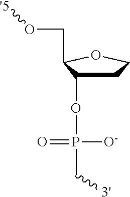

(for incorporation at 5′end, internally, or 3′ end), a PC Spacer(

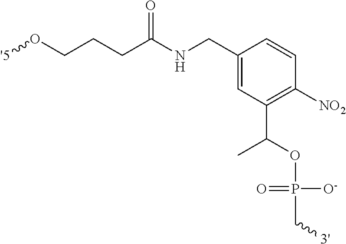

(for incorporation at the 5′ end or internally), a Spacer 9 (a thriethylene glycol spacer

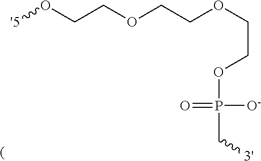

for incorporation at the 5′ end, internally, or 3′ end)), a Spacer 18 (18-atom hexa-ethyleneglycol spacer, or

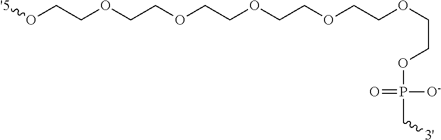

incorporation at the 5′ end, internally, or 3′ end)).

In some examples, the blocking group may be provided at the 3′ end of the primer. In some examples, the blocking group may be provided at the 3′ end of a mismatching DNA. In some examples, the blocking group may be provided within the one or more matching DNA bases and at the 5′ end of the mismatching DNA base. In some examples, where high fidelity of template amplification is desired, the blocking group is provided within the one or more matching DNA bases at the 5′ end of the mismatching DNA bases.

In some examples, the primer and/or probe may comprise formula (I):

Dₙ₁-Rₙ₂-Dₙ₃-Mₙ₄-D_{n3-X} (I)
wherein
D is a DNA base that match target nucleic acid,
R is an RNA base that matches target,
M is a DNA base that is a mismatch to target nucleic acid,
X is a blocking group,
n1 is an integer representing the number of bases of between 1 to 20,
n2 is an integer representing the number of bases of between 1 to 5, and
n3 is an integer representing the number of bases of between 0 to 5.

In some examples, the primer and/or probe may comprise, in order from 5′ to 3′, a functional primer, a cleavage site, one or more matching DNA bases, one or more mismatch DNA base, and one or more blocking group.

In some examples, the primer and/or probe may comprise, in order from 5′ to 3′, a functional primer, a cleavage site, one or more matching DNA bases, one or more blocking group, one or more matching DNA bases, and one or more mismatching DNA.

In some examples, the primer comprising one or more RNA bases is an rhPCR primer (i.e. an RNase-dependent PCR primers), optionally the RNase-dependent PCR primers is an RNase H-dependent PCR primers. When the primer is an RNase H-dependent PCR primers, which is described in US 2015/225782 A1, the content of which is incorporated herein by reference.

The inventors of the present disclosure found that the combination of amplification of the target nucleic acid in surfactant (i.e. emulsion based PCR) with the oligonucleotide having cleavage site (i.e. rhPCR primers) advantageously increases the specificity of the amplification method.

In some examples, the present disclosure also includes the use of a probe. In some examples, the probe may be an oligonucleotide attached/conjugated to a detectable agent (such as a fluorophore and/or quencher). In some examples, the probe may be an oligonucleotide attached/conjugated to a detectable agent (such as a fluorescent label and/or quencher) and a groove binder. In some examples, the probe may comprise a nucleic acid binding reagent (such as SYBR® Green dye).

In some examples, the control nucleic acid is added to the sample at a constant amount to thereby normalizes of the amplification efficiency across a plurality of samples, optionally the control nucleic acid is added to the sample at about 102 to 1010 copies.

In some examples, the control nucleic acid (i.e. spike-in controls) does not compete or interfere with the amplification of the target nucleic acid. In some examples, the controls have low sequence homology to the target nucleic acid (for example it has low sequence homology to any human genes). In some examples, the control nucleic acid has a different sequence from the target nucleic acid. In some examples, the control nucleic acid is nucleic acid that cannot be found in the sample (i.e. exogenous from the sample) and/or is not a housekeeping gene. In some examples, the control nucleic acid is included in greater abundance than the target nucleic acid. The addition of a control nucleic acid allows for normalization of the technical amplification efficiency across samples. The control nucleic acid also advantageously normalizes for any unintended variation in the experiment.

In some examples, the control nucleic acid may be a DNA and/or RNA. In some examples, the control nucleic acid may be substantially no or very low sequence homology or substantially different from human gene. In some examples, the control nucleic acid may be luciferase. In some examples, the control nucleic acid may be luciferase RNA. As illustrated in the Experimental Section, the methods as disclosed herein may include the usage of luciferase RNA as a spiked—in for normalising PCR efficiency.

Without wishing to be bound by theory, it is believed that the method as disclosed herein may leverage on the “CoT effect” that increases the sensitivity of a method with minimal loss in linearity when used in quantitative methods. As used herein, the “CoT effect” refers to an amplification method where the presence of greater abundance of a particular nucleic acid results in a systemic bias against the more abundant of the two PCR products (one being an abundant nucleic acid (may be an internal control or an endogenous nucleic acid present in abundance in the sample) and the other being the target nucleic acid). The slowdown in amplification of abundant products allows the target nucleic acid/target of interest (which may be present in less quantity) to become more visible in the fingerprint. It is believed that the increase visibility of the target nucleic acid/target of interest (which may be present in lesser quantities) allow the target of interest (such as rarer cDNAs) to be sampled more efficiently. In another word, CoT PCR enable selective amplification of low concentration DNA resulting in increase of sensitivity for downstream applications.

In some examples, the methods as disclosed herein may comprise CoT PCR. In some examples, the amplification step in the method as described herein includes interposing an annealing step between denaturation and priming. Without wishing to be bound by theory, since the amount of re-annealing depends on the product of the initial concentration and time, CoT, the more abundant a sequence the greater will be the extent of its conversion to the double-stranded form. These hybrids will then fail to be copied in the polymerisation step of the cycle. Thus, as each sequence reaches the threshold concentration at which considerable re-annealing occurs it ceases to be exponentially amplified, and in this way, all sequences will eventually have the same concentration. In some examples, the CoT PCR may be as described by Brenner S. and Jones DSC, 1972 (Wellcome collection, which can be accessed here: https://wellcomecollection.org/works/h39jksrt/items, the content of which is incorporated herein).

In some examples, the method comprises a subsequent amplification step (e.g., a second or third or more thermocycling reactions) with enzyme activation, denaturation, hybridization, annealing and extension.

Advantageously, CoT PCR enrichment may preferentially amplify rare amplicons over abundant ones by taking advantage of the CoT effect.

In a comparative example, a combination of emulsion rhPCR and CoT is shown to decrease the number of PCR cycles required for detection of a target polynucleotide as compared to emulsion rhPCR without CoT. Thus, a combination of emulsion rhPCR and CoT may advantageously increase a sensitivity of embodiments of the method in determining, detecting or quantifying a pancreas-associated polynucleotide. This allows embodiments of the method to detect or quantify low levels of pancreas-associated polynucleotide in a subject, which may not be possible otherwise.

Without wishing to be bound by theory, it is believed that the Cot effect is described by a faster rate of re-association of higher abundance single stranded DNA (ssDNA) to double-stranded DNA (dsDNA). Using this phenomenon, one way to enrich for rare target is to allow the higher abundant ssDNA to reassociate to dsDNA and then follow up with a method to remove the dsDNA to achieve removal of abundant dsDNA. In the present disclosure however, embodiments of the method do not involve removing the abundant dsDNA that was amplified (e.g., by rhPCR). Instead, the less abundant DNA in the reaction is allowed to have a higher probability of amplified. To achieve this, the CoT phenomenon may be implemented during amplification (e.g., during rhPCR) by adjusting/controlling the thermal cycling profile of the amplification process (e.g., rhPCR). The PCR reaction mix (e.g., the rhPCR reaction mix) may be held at the melting temperature of the dsDNA amplicons during the denaturing step of the PCR (e.g., rhPCR). Without wishing to be bound by theory, it is believed that, when the PCR reaction is being held at the melting temperature of the dsDNA amplicon, the CoT effect kicks in: the abundant dsDNA preferentially remains double stranded, and only dsDNA amplicons at low concentration will dissociate. These dissociated ssDNA are the only ones accessible to primers in the subsequent annealing and extension step which completes the PCR. By repeating this every cycle (i.e., holding the PCR reaction at the melting temperature of the dsDNA amplicon at the denaturing step), the initial low abundance amplicons will amplify up to a point where it becomes suitably abundant and joins other high abundance amplicons and be inhibited from disassociation, which allows for other remaining low abundance amplicons to be amplified.

In some examples, the control nucleic acid is a nucleic acid that may be added to the method as disclosed herein in a fixed amount (or a constant amount in all samples). In some examples, the control nucleic acid is provided at a concentration that is higher than the predicted concentration of the target of interest (or target nucleic acid).

In some examples, the amount of control nucleic acid added to the sample is about 10² to 10¹⁰ copies. In some examples, the amount of control nucleic acid may be about 100 copies, 10³ copies, 10⁴ copies, 10⁵ copies, 10⁶ copies, 10⁷ copies, 10⁸ copies, 10⁹ copies, or 10¹⁰ copies. In some examples, the amount of control nucleic acid may be about 100 to 10⁹ copies, or about 100 to 10⁸ copies, or about 100 to 10⁷ copies, or about 100 to 10⁶ copies, or about 100 to 10⁵ copies. In some examples, the amount of control nucleic acid is about 10⁵ copies.

In some examples, the method may further comprise the detection of a second control nucleic acid that is present endogenously in the sample. For example, the second control nucleic acid may be a housekeeping gene. In some examples, the second control nucleic acid may include, but is not limited to, actin beta (ACTB), glyceraldehyde 3-phosphate dehydrogenase (GAPDH), ribosomal protein S18 (RPS18), ubiquitin C (UBC), beta-2 microglobulin (B2M), glucuronidase beta (GUSB), hypoxanthine-guanine phosphoribosyltransferase (HPRT), phosphoglycerate kinase 1 (PGK1), peptidylprolyl isomerase A (PPIA), TATA box binding protein (TBP), transferrin receptor (TFRC), tyrosine 3-monoxygenase/tryptophan 5-monooxygenase activation protein zeta (YWHAZ), tubulin, heat shock protein 90 (HSP90), hypoxanthine guanine phosphoribosyl transferase (HPRT), succinate dehydrogenase complex, subunit A (SDHA), mitochondrially encoded 12S ribosomal RNA (mtRNR1), mitochondrially encoded 16S RNA (mtRNR2), and the like. The detection of the second control nucleic acid allows for the normalization of extraction efficiency across samples.

In some examples, the method further comprises analysing data by normalizing raw values (such as Ct value) to the levels of control nucleic acids (such as housekeeping gene or spiked in luciferase RNA). In some examples, the method comprises contacting the target nucleic acid with an annealing reagent comprising a primer of the target nucleic acid, a control nucleic acid, and an annealing mixture (including deoxyribonucleotide triphosphate (dNTP) mixes). In some examples, where the nucleic acid is an RNA, the method comprises contacting the target nucleic acid with an annealing reagent comprising a reverse primer of the target nucleic acid, a control nucleic acid, and an annealing mixture (including deoxyribonucleotide triphosphate (dNTP) mixes).

In some examples, the annealing step precede the reverse transcription and amplification cycles.

In some examples, the method further comprises subjecting the target nucleic acid to reverse transcription. In some examples, the method further comprises a reverse transcription of the target nucleic acid after annealing step.

In some examples, the method comprises contacting the target nucleic acid with a reverse transcription agent comprising a reverse transcriptase, and a reverse transcriptase mixture (including DTT).

In some examples, the method further comprises inactivation of the reverse transcriptase.

In some examples, the method further comprises a step of quantifying the amount of target nucleic acid present in the sample and/or sequencing the target nucleic acid in the sample.

In some examples, the method of the present disclosure may be adaptable to include processing where amplified cDNA exhibits compatibility for downstream further processing. This is because the method of the present disclosure advantageously provides an adaptable end point where amplified cDNA exhibit compatibility for downstream quantification using methods known in the art. For example, the cDNA as amplified by the method as disclosed herein may be used in further steps of quantifying the amount of target nucleic acid by performing quantitative real-time PCR, next generation sequencing, UV absorbance with spectrophotometer, fluorescence dyes, agarose gel electrophoresis, microfluidic capillary electrophoresis, diphenylamine method, droplet digital PCR, and the like.

Diverse RNA transcripts are widely detected to be circulating within the human plasma. The notion of using these circulating cell free RNA (cfRNA) as potential biomarkers has recently emerged from comprehensive assessments using high throughout sequencing technologies that led to the identification of tissue-specific cfRNA changes that correlate to pathological conditions such as cancer and metabolic diseases. Follow up work by the inventors have also shown that tissue specific cfRNA provides a non-invasive window for studying hard to reach tissues under different biological conditions.

Quantification of tissue-specific cfRNA is expected to vary based on the tissue of origin and the biological state of the cells when releasing cfRNA via apoptosis. However, the lack of effective and sensitive molecular tools to amplify and quantitate RNA biases against low abundance tissue specific cfRNA of interest. This in turn limits the widespread use of cfRNA as biomarkers. To address this, the present disclosure discloses a molecular protocol that overcome this by combining emulsion-based PCR together with specifically designed rhprimers that pre-amplifies tissue specific cfRNA for downstream quantitation with qPCR, or next generation sequencing.

Therefore, in some examples, the nucleic acid is a cell free nucleic acid, optionally a circulating cell free nucleic acid. In some examples, the cell free nucleic acid is a cell free DNA and/or a cell free RNA. In some examples, the nucleic acid is a circulating cell free RNA. In some examples, the cell free nucleic acid is an isolated cell free nucleic acid.

In some examples, when the nucleic acid is an RNA, the method comprises annealing the target nucleic acid is in the presence of a reverse primer of the target nucleic acid and the control nucleic acid, subjecting the annealed sample to reverse transcription, and subjecting the target nucleic acid to one or more amplification step in the presence of a mixture comprising a surfactant and a primer comprising one or more RNA base and a cleavable 3′ end.

In some examples, the reverse primers of the target nucleic acid may comprise about 10 to 40 bases, or 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, 30 bases, 35 bases, or 40 bases. In some examples, the reverse primers of the target nucleic acid may be about 15-18 bases, or about 16 bases.

In some examples, the method further comprises the extraction of the nucleic acid from a sample.

In some examples, the sample may include any items that may contain nucleic acid of interest. For example, the items may be a surface of an equipment, a laboratory bench, a public surface (such as, but not limited to, surface on an elevator/lift/doorknobs/toilet, surface on a public transport, surface of airport areas, surface of school areas, surface of shopping mall or supermarket areas, surface of restaurants/hawkers/cafes, and the like), frequently touched surfaces adjacent to patients in hospitals/clinics (such as, but not limited to, areas adjacent to or at the hospital bed, hospital/clinic waiting areas, quarantine rooms and the like).

In some examples, the sample may be a biological sample. In some examples, the nucleic acid is obtained from a biological sample.

In some examples, the samples may be obtained at different time points of the disease state. For examples, the disease state may include pre-surgery, peri-operative period, immediately after surgery, short term post-surgery, long-term post-surgery, antibody positive state, recurrent or persistent cancer, and the like.

The presence or absence of a target nucleic acid can be measured quantitatively or qualitatively. Target nucleic acid can come in a variety of different forms including, for example, simple or complex mixtures, or in substantially purified forms. For example, a target nucleic acid can be a part of a sample that contains other components or can be the sole or major component of the sample. Therefore, a target nucleic acid can be a component of the circulatory system (such as blood, serum, plasma, or combinations thereof), a whole cell or tissue, a cell or tissue extract, a fractionated lysate thereof or a substantially purified molecule. In some examples, the target nucleic acid is a region of interest in a cell free DNA and/or RNA. In some examples, the target nucleic acid is a region of interest in a cell free RNA. In some examples, the target nucleic acid is a region of interest in a circulating cell free RNA.

In some examples, the method detects the presence and/or absence of any one of the following interest, such as, but not limited to, a pathogen, a disease, a cancer, a genetic defect, and the like. For example, pathogens may be a bacterial pathogen, a viral pathogen, a fungal pathogen, or a parasite.

Examples of a bacterial pathogen may include, but is not limited to, Escherichia coli, Mycobacteria spp, Salmonella spp, Staphylococcus spp, Clostridium difficile, Listeria monocytogenes, Group B streptococci, vancomycin-resistant enterococci (VRE), and the like.

Examples of a viral pathogen may include, but is not limited to, Human papillomavirus, Rhinovirus, Human cytomegalovirus in HIV-1 positive patient, Hepatitis virus, Coronavirus (CoV), severe acute respiratory syndrome (SARS), monkey pox virus and the like.

Examples of a fungal pathogen may include, but is not limited to, Botrytis cinerea, Pseudomonas syringae, Fusarium oxysporum and the like.

Examples of a parasite may include, but is not limited to, Leishmania parasites, Giardia, Cryptosporidium, Entamoeba and the like.

In some examples, the disease may be a metabolic disorder, such as, but is not limited to, hypothyroidism, hyperthyroidism, diabetes, mitochondrial disorders, phenylketonuria (PKU), and the like.

In some examples, the cancer may include, but is not limited to, thyroid cancer, pancreatic cancer, breast cancer, colon cancer, lung cancer, liver cancer, skin cancer, and the like.

In some examples, the genetic defects may include, but is not limited to, a prenatal genetic defect, Cystic fibrosis, and the like. In some examples, the prenatal genetic defect may include, but is not limited to, Down syndrome (Trisomy 21), Turner Syndrome, Edwards' syndrome, and the like.

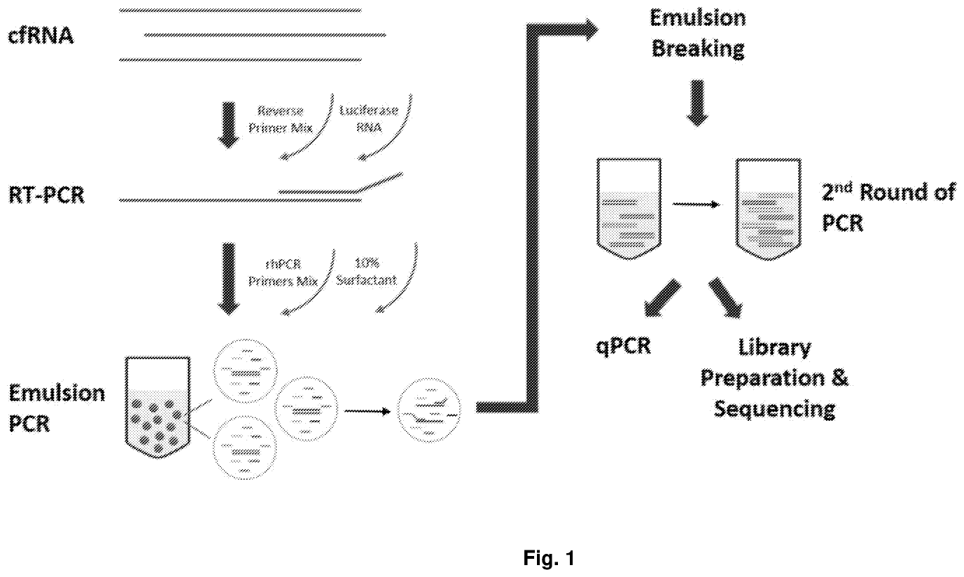

The present invention can advantageously be performed as a “one-pot amplification” process. The one-pot amplification made possible by the use of the emulsion PCR and the spiking of the sample with a control nucleic acid. As illustrated in FIG. 1, the methods as disclosed herein can be performed in a single closed tube from annealing step to reverse transcription and amplification step.

In some examples, the method is a real time amplification method.

In some examples, the method as disclosed herein may be performed on samples drawn at multiple time points. In some examples, the samples may be drawn/obtained from the subject at one or more, 2 or more, 3 or more, 4 or more, 5 or more, 6 or more, 7 or more, 8 or more, 9 or more, or 10 or more time points. In some examples, the samples may be drawn/obtained from the subject at 1 to 10 time points, or at least at 1 time point, or at least at 2 time points, or at least at 3 time points, or at least at 4 time points, or at least at 5 time points, or at least at 6 time points, or at least at 7 time points, or at least at 8 time points, or at least at 9 time points, or at least at 10 time points. In some examples, the samples may be drawn/obtained from the subject at at least 5 time points. For example, the 5 time points may include pre-surgery, short-term post-surgery, long-term post-surgery, and the like.

In another aspect, there is provided a nucleic acid amplification mixture comprising a first mixture comprising: a control nucleic acid, and a second mixture comprising: a surfactant, and an oligonucleotide primer and/or probe capable of hybridizing with a target nucleic acid, wherein the oligonucleotide primer and/or probe comprises a cleavage site and a cleavable 3′ end.

In some examples, the second mixture further comprises amplification reagents.

In some examples, wherein the amplification agent comprises detectable primers and/or probes.

In some examples, there is provided a kit comprising the reagents and/or mixtures used in the methods as disclosed herein.

In some examples, the target nucleic acid may be present in the sample in minute amount or in low quantity. In some examples, the target nucleic acid may not be present in abundance.

In some examples, the amount of sample nucleic acid may be about 1 μL to about 100 μL. In some examples, the amount of cfRNA in the sample may be about 1 μL to 90 μL, or about 5 μL to about 80 μL, or about 10 μL to about 50 μL. In some examples, the amount of cfRNA in the sample may be no more than 50 L, no more than 40 μL, no more than 30 μL, no more than 20 μL, no more than 19 μL, no more than 18 μL, no more than 17 μL, no more than 16 μL, no more than 15 μL, no more than 14 μL, no more than 13 μL, no more than 12 μL, no more than 11 L, no more than 10 μL, and the like.

In some examples, the amount of sample nucleic acid may be about 500 picogram (pg) to about 1000 μg. In some examples, the amount of sample nucleic acid may be about 500 pg, may be about 550 pg, may be about 600 pg, may be about 650 pg, may be about 700 pg, may be about 750 pg, may be about 800 pg, may be about 850 pg, may be about 900 pg, may be about 950 pg, may be about 1000 pg, may be about 1050 pg, may be about 1100 pg, may be about 1150 pg, may be about 1200 pg, may be about 1300 pg, may be about 1400 pg, may be about 1500 pg, may be about 2000 pg, may be about 3000 pg, may be about 4000 pg, may be about 5000 pg, may be about 6000 pg, may be about 7000 pg, may be about 8000 pg, may be about 9000 pg, may be about 10,000 pg, may be about 15,000 pg, may be about 20,000 pg, may be about 25,000 pg, may be about 30,000 pg, may be about 35,000 pg, may be about 40,000 pg, may be about 50,000 pg, may be about 100,000 pg, may be about 0.01 μg, may be about 0.02 μg, may be about 0.03 μg, may be about 0.04 μg, may be about 0.05 μg, may be about 0.06 μg, may be about 0.07 μg, may be about 0.08 μg, may be about 0.09 μg, may be about 0.1 μg, may be about 0.2 μg, may be about 0.3 μg, may be about 0.4 μg, may be about 0.5 μg, may be about 0.6 μg, may be about 0.7 μg, may be about 0.8 μg, may be about 0.9 μg, or may be about 1 μg. In some examples, the amount of sample nucleic acid may be no more than 2 μg, 3 μg, 4 μg, 5 μg, 6 μg, 7 μg, 8 μg, 9 μg, 10 μg, 11 μg, 12 μg, 13 μg, 14 μg, 15 μg, 20 μg, 30 μg, 40 μg, 50 μg, 100 μg, 150 μg, 200 μg, 300 μg, 400 μg, or 500 μg, or 2 to 1000 μg.

In another aspect, there is provided a method of detecting and/or determining the presence and/or the amount of a target nucleic acid comprising annealing the target nucleic acid in the presence of a control nucleic acid, and subjecting the target nucleic acid to one or more amplification step in the presence of a mixture comprising a surfactant and an oligonucleotide capable of hybridizing to the target nucleic acid comprising one or more RNA base and a cleavable 3′ end.

Also disclosed are methods of quantifying tissue/organ health in disease states. For example, the methods as disclosed herein may be applied to metabolic disorders and cancer surveillance.

As such, the present disclosure provides sensitive and multiplex methods for targeted amplification and/or quantification of low amounts of naturally occurring tissue specific RNA extracted from plasma.

In some examples, the method may comprise: (a) providing a reaction mixture comprising (i) rhPCR primers (e.g. an oligonucleotide primer having a cleavage domain positioned 5′ of a blocking group and 3′ of a position of variant, the blocking group linked at or near the end of the 3′-end of the oligonucleotide primer wherein the blocking group prevents primer extension and/or inhibits the oligonucleotide primer from serving as a template for DNA synthesis), (ii) a control RNA, (b) subjecting the reaction mixture to reverse transcription conditions, (c) contacting the resulting mixture from step (b) with a surfactant and an amplification mixture, (d) subjecting the mixture from step (c) to amplification conditions sufficient to result in the amplification of the nucleic acid.

In some examples, there is provided a method of amplification of a target nucleic acid, the method comprising annealing the target nucleic acid in the presence of a reverse primer of the target nucleic acid and a control nucleic acid, subjecting the target nucleic acid to reverse transcription, and subjecting the target nucleic acid to one or more amplification step in the presence of a mixture comprising a surfactant and a primer comprising one or more RNA base and a cleavable 3′ end.

### EXPERIMENTAL SECTION

Reverse Transcription PCR (RT-PCR) of cfRNA

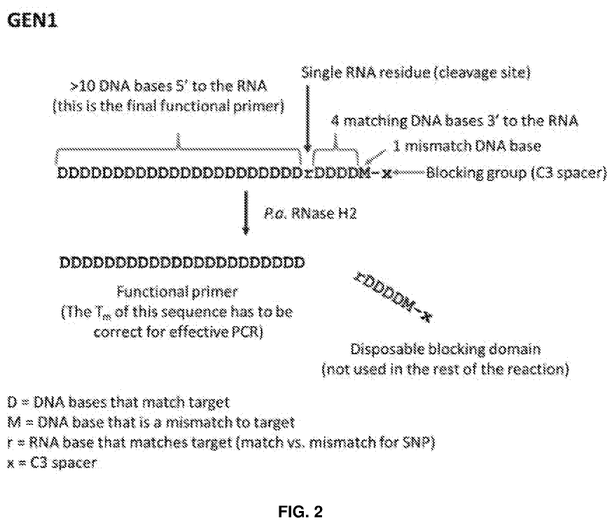

10 μL of extracted cfRNA was annealed with 0.08 UM of reverse primers mix of the targets of interest, 10⁵ copies of Luciferase control RNA (Promega, Cat no. L4561) and 0.4 mM of dNTP mix (Thermofisher Scientific, Cat no. R0191) at 66° C. for 5 minutes. Reverse transcription of cfRNA was performed using 100U Superscript™ III Reverse Transcriptase (Invitrogen, Cat no. 18080044) and 0.5 μL of 0.1 M DTT at 25° C. for 5 minutes, 50° C. for 50 minutes, followed by enzyme inactivation at 95° C. for 3 minutes. cDNA from the reverse transcription was added to the PCR mixture with 2.5U Platinum™ Taq DNA Polymerase (Invitrogen, Cat no. 10966), 0.4 mM dNTP mix, 1.5 mM Magnesium Chloride, 0.5 UM of rhPCR primers mix (Integrated DNA Technologies, GEN1, FIG. 2) of the targets of interest and 26 mU of RNase H2 enzyme (Integrated DNA Technologies, Cat no. Nov. 2, 2012-01).

### Emulsion PCR and Emulsion Breaking

Emulsion was generated by adding 3 parts of 10% 008-FluoroSurfactant (RAN Biotechnologies) in 3M Fluorinert™ Engineered Fluid (3M, Cat no. FC-40) to 1 part of PCR reaction mixture. The mixture was vortexed until uniform turbidity. Thermocycling reaction includes enzyme activation at 94° C. for 2 minutes, followed by 20 cycles of denaturation (94° C., 15 seconds), annealing (61° C. 30 seconds), and extension (68° C., 1 minute).

Reaction was frozen at −80° C. for 1 hour before thawing at room temperature. The top fraction containing the reaction mix was transferred to a fresh tube. Fraction that was recovered was topped up with the same amount of Platinum™ Taq polymerase and RNase H2 enzyme used in the previous PCR reaction.

Second thermocycling reaction started with enzyme activation at 94° C. for 2 minutes, followed by 20 cycles of denaturation (94° C., 15 seconds), hybridization (78° C., 10 minutes), annealing (61° C., 30 seconds), and extension (68° C., 1 minute). Residual primers were removed with 30U of RecJf (New England Biolabs, Cat no. M0264) at 37° C. for 1 hour, followed by enzyme inactivation at 80° C. for 20 minutes. The reaction can be used for downstream quantification using qPCR and next generation sequencing.

### RESULTS

### Validation of the Cot-Emulsion PCR Molecular Technology Protocol Using Artificial Spike in Controls

Luciferase RNA (LUC), which is not found in normal plasma RNA, is used as a spiked—in control to mimic the presence of low level of circulating RNA of interest across different applications. A range of LUC copies are used: 216 [65536 molecules], 218 molecules], 222 [4, 194,304 molecules] are spiked into RNA extracted from 1 ml of human plasma. These range of spiked-ins are used to illustrate the range of operability as well as scalability of the protocol.

These spiked in samples are put through 2 different versions of the molecular protocol. The first protocol comprises all the major steps including CoT amplification, to validate that the molecular technology is detecting the spike in LUC molecules. The second protocol has the CoT amplification process removed, to validate and illustrate the impact of CoT amplification in improving the Ct measurements and sensitivity of detection. In addition to LUC, housekeeping genes [RPS18, ACTB] are also used as positive controls for the platform.

The inventors of the present disclosure show that the current protocol amplifies the target spiked in LUC and the quantified Ct cycles scales with the input range of molecules. In addition, CoT amplification significantly improves the sensitivity of the protocol by decreasing the Ct cycles.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural, electrical and optical changes may be made without deviating from the scope of the invention. Example embodiments are not necessarily mutually exclusive as some may be combined with one or more embodiments to form new exemplary embodiments. The example embodiments should not be construed as limiting the scope of the disclosure.

FIG. 1 shows a schematic diagram of the workflow of reverse transcription and amplification of circulating RNA for downstream quantification using qPCR and next-generation sequencing, cfRNA, spiked with Luciferase RNA control, was reverse transcribed with Superscript™ III Reverse Transcriptase (Invitrogen, Cat no. 18080044). The product was amplified with rhPCR primers and Platinum™ Taq DNA Polymerase (Invitrogen, Cat no. 10966) using emulsion PCR. The residual primers were removed with RecJf (New England Biolabs, Cat no. M0264). Amplified products were used for qPCR quantification and sequencing.

FIG. 2 shows the design of a rhPCR primer as used in the present disclosure. In one embodiment, the rhPCR primer may consists of five different parts: starting from the 5′ end with a functional primer, the cleavage site, four matching DNA bases, one mismatch DNA base, and a blocking group at the 3′ end. Cleavage of the RNA residue by RNase H2 will take place when the primer is perfectly complementary to the template, releasing the 3′ blocking group.

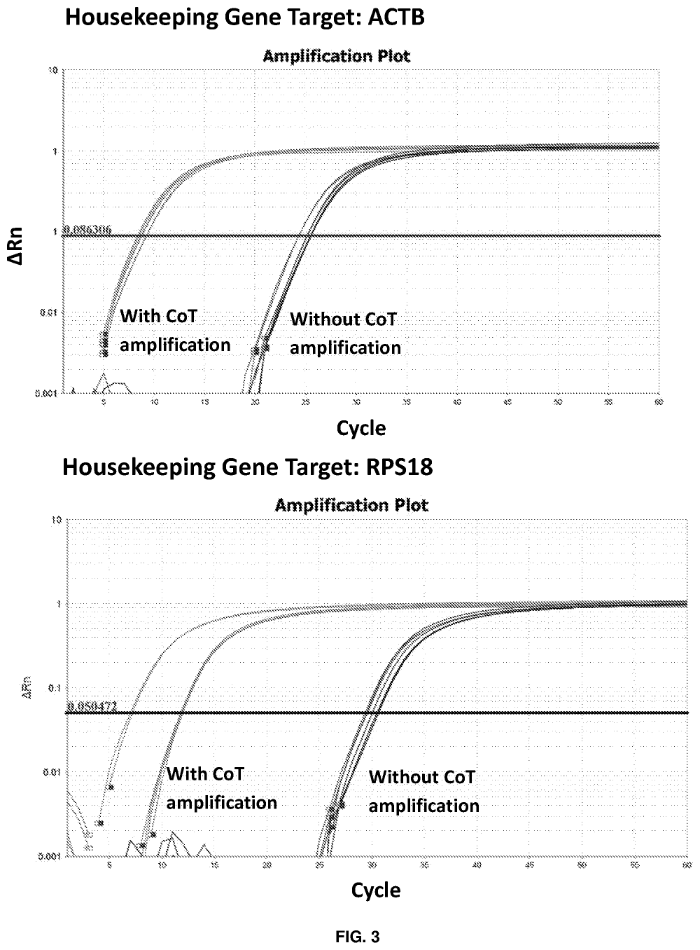

FIG. 3 shows Ct curves of housekeeping genes validating the protocol in amplification of housekeep genes found in plasma. Each curve is a biological replicate. Addition of CoT amplification decreases the CT cycle and improves sensitivity.

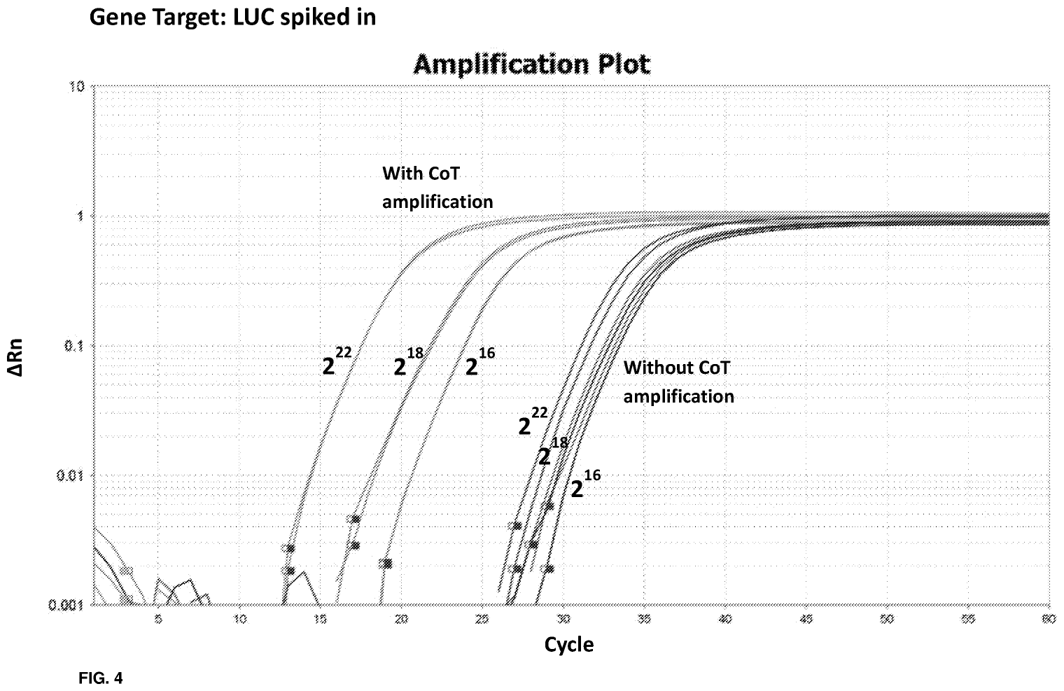

FIG. 4 shows Ct curves of LUC spiked-ins across a range of spiked in molecules. Each curve illustrates a different LUC spiked in. Addition of CoT amplification decreases the CT cycle and improves sensitivity.

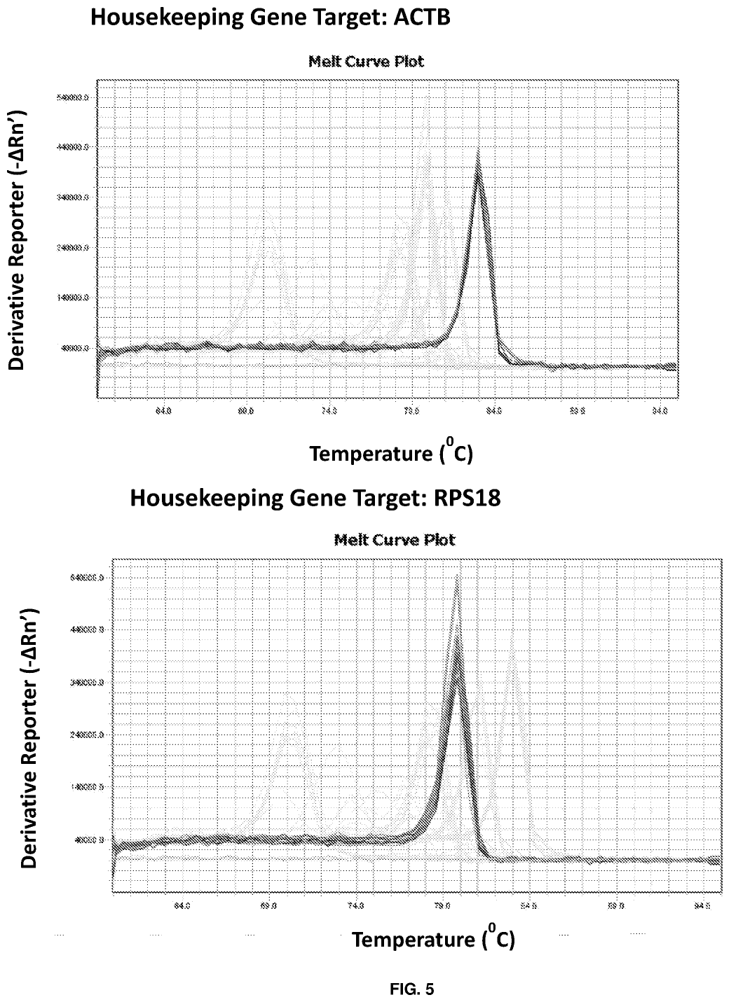

FIG. 5 shows melt curves of housekeeping genes validating the protocol in amplification of housekeep genes found in plasma. Each curve is a biological replicate. Sharp peaks indicating strong and specific amplification of the target at the intended melting temperature.

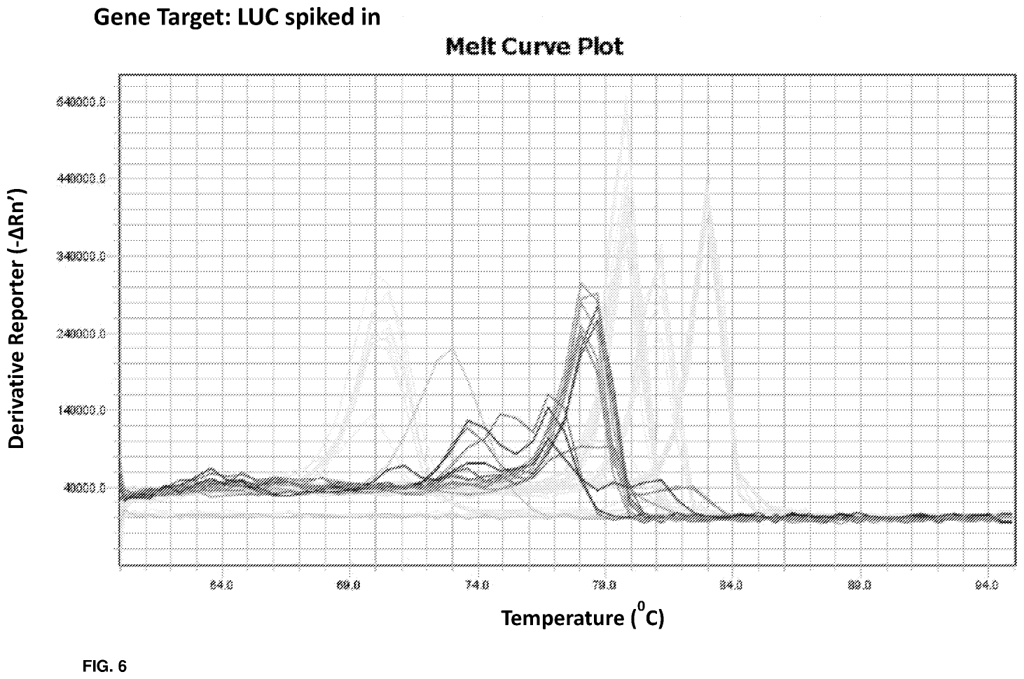

FIG. 6 shows melt curves of LUC spike-in targets validating the protocol in amplification of housekeep genes found in plasma. Each curve is a biological replicate. Sharp peaks indicating strong and specific amplification of the target at the intended melting temperature.

### APPLICATIONS

Embodiments of the methods disclosed herein provide a fast and efficient way of amplifying a target nucleic acid that can be found in a sample only in small amounts. Embodiments of the disclosed methods also seek to overcome the problems of providing a method of amplifying a target nucleic acid with increased sensitivity.

Advantageously, the methods and/or mixtures as disclosed herein provides a one pot amplification of low input nucleic acid (such as RNA) using a combination emulsion based PCR amplification using specific primer/probe design (such as rhPCR primer design).

Even more advantageously, the methods and/or mixtures as disclosed herein provides a robust non-chemical-based method of recovering emulsion PCR product using freeze-thaw cycle.

The present disclosure also advantageously provides for the inclusion of quality control method of normalizing PCR efficiency that utilizes spiked in luciferase RNA.

The present disclosure also provides an amplification (such as PCR) cycling protocol that leverages the CoT effect for increased sensitivity with minimal loss in linearity using in quantitation.

The present disclosure also provides an adaptable end point where amplified cDNA exhibits compatibility for downstream quantification using either qPCR or next-generation sequencing

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of amplification of a target nucleic acid, the method comprising:
annealing the target nucleic acid in the presence of a control nucleic acid, and
subjecting the target nucleic acid to one or more amplification step in the presence of a mixture comprising a surfactant and an oligonucleotide primer and/or probe capable of hybridizing with the target nucleic acid, wherein the oligonucleotide primer and/or probe comprises a cleavage site and a cleavable 3′ end.

2. The method of claim 1, wherein the amplification step of the nucleic acid is performed in the presence of three parts surfactant to one part amplification mixture.

3. The method of claim 1, wherein the method comprises two amplification steps.

4. The method of claim 1, wherein the method further comprises a step of freeze and thawing the amplified mixture.

5. The method of claim 1, wherein the method further comprises a step of freeze and thawing the amplified mixture between the one or more amplification steps.

6. The method of claim 1, wherein the amplification step includes interposing an annealing step between denaturation and priming.

7. The method of claim 1, wherein the oligonucleotide primer and/or probe capable of hybridizing with the target nucleic acid comprises a 5′ end of a functional primer, a cleavage site, one or more matching DNA bases, and one or more mismatch DNA base with one or more blocking group at the 3′ end.

8. The method of claim 1, wherein the cleavage site is one or more RNA residues.

9. The method of claim 1, wherein the oligonucleotide primer and/or probe capable of hybridizing with the target nucleic acid comprises a 5′ end of a functional primer, a cleavage site consisting of one or more RNA residue, one or more matching DNA bases, and one or more mismatch DNA base with one or more blocking group at the 3′ end.

10. The method of claim 1, wherein the method comprises the step of cleaving the oligonucleotide primer and/or probe with an RNase enzyme.

11. The method of claim 1, wherein the control nucleic acid is added to the sample at a constant amount to thereby normalizes of the amplification efficiency across a plurality of samples, optionally the control nucleic acid is added to the sample at about 10² to 10¹⁰ copies.

12. The method of claim 1, wherein the method further comprises a reverse transcription of the target nucleic acid after annealing step.

13. The method of claim 1, wherein the method further comprises a step of quantifying the amount of target nucleic acid present in the sample and/or sequencing the target nucleic acid in the sample.

14. The method of claim 1, wherein the target nucleic acid is a cell free nucleic acid, optionally a circulating cell free nucleic acid.

15. The method of claim 1, wherein the target nucleic acid is a cell free RNA, optionally a circulating cell free RNA.

16. The method of claim 1, wherein the target nucleic acid is obtained from a biological sample.

17. The method of claim 1, wherein when the target nucleic acid is an RNA, the method comprises:
annealing the target nucleic acid is in the presence of a reverse primer of the target nucleic acid and the control nucleic acid,
subjecting the annealed sample to reverse transcription, and
subjecting the target nucleic acid to one or more amplification step in the presence of a mixture comprising a surfactant and an oligonucleotide primer and/or probe capable of hybridizing with the target nucleic acid, wherein the oligonucleotide primer and/or probe comprises a cleavage site and a cleavable 3′ end.

18. The method of claim 1, wherein the method is a real time amplification method.

19. A nucleic acid amplification mixture comprising:
a first mixture comprising:
a control nucleic acid, and
a second mixture comprising:
a surfactant, and
an oligonucleotide primer and/or probe capable of hybridizing with a target nucleic acid, wherein the oligonucleotide primer and/or probe comprises a cleavage site and a cleavable 3′ end.

20. A method of detecting and/or determining the presence and/or the amount of a target nucleic acid comprising:
annealing the target nucleic acid in the presence of a control nucleic acid, and
subjecting the target nucleic acid to one or more amplification step in the presence of a mixture comprising a surfactant and an oligonucleotide capable of hybridizing to the target nucleic acid comprising one or more RNA base and a cleavable 3′ end.
